(19) United States
(12) Patent Application Publication
(10) Pub. No.: US 2004/0023882 A1
(43) Pub. Date: Feb. 5, 2004

(54) PTH derivatives resistant to skin proteases

(76) Inventors: Krishna G. Peri, Montreal (CA); Kim High, St-Lazare (CA); Annie Bergeron, Repentigny (CA); Serge Moffett, St-Caurent (CA); Thierry Abribat, Montreal (CA)

Correspondence Address: Michael R. Krawzsenek, FULBRIGHT & JAWORSKI L.L.P., Suite 2400, 600 Congress Avenue, Austin, TX, 78701, US

(21) Appl. No.: 10/440,473

(22) Filed: May 16, 2003

Related U.S. Application Data
(60) Provisional application No. 60/378,072, filed on May 16, 2002.

(51) Int. Cl. A61K 38/17; A61K 7/28; C07K 14/00

(52) U.S. Cl. 514/12; 530/324; 424/50

(57) ABSTRACT

Protease-resistant analogues of biologically active derivatives of human PTH are described. These analogs are intended for use in therapeutic preparations for the treatment of various medical conditions in which bone loss is encountered or is susceptible of being encountered. The analogs specified are hPTH(1-34) and hPTH(1-31). More particularly, protease-resistant analogs of PTH adapted for transdermal administration are described.

The present invention claims benefit of priority to U.S. Provisional Serial No. 60/378,072, filed May 16, 2002, the entire contents of which is incorporated by reference.

### FIELD OF THE INVENTION

The present invention relates to Parathyroid Hormone (PTH) derivatives resistant to skin proteases.

### BACKGROUND OF THE INVENTION

Parathyroid hormone (1-84) secreted by the parathyroid gland is processed to release N- and C-terminal fragments in circulation. PTH is involved, along with calcitonin, vitamin D, and the calcium sensing receptor, in calcium homeostasis and displays potent anabolic and catabolic actions on cancellous bone. The actions of PTH are mediated by the PTHR1 and PTHR2 receptors. Though the role of PTHR1 in bone turnover, in differentiation and function of osteoblasts, osteoclasts and osteocytes, and in renal calcium handling is well known, the physiological role of PTHR2 is yet to be delineated. Even though the actions of PTH (1-84) via PTH 1 are reproduced equipotently by the N-terminal truncated product, PTH(1-34), the physiological roles of the carboxy-terminal fragment of PTH(1-84) are not fully understood (For a review see Whitfield, J. F. et al. 2000. Medscape Women's Health; 5(5); Whitfield, J. F. et al. 1998. The parathyroid hormone: an unexpected bone builder for treating osteoporosis; Landes Bioscience Co. Autin, Tex.).

PTH has been shown to be a potent osteo-anabolic agent in animal models as well as in human clinical trials (Meer, R. M. et al. 2001, New Engl. J. Med. 344(19): 1434 and references cited therein). There is a great deal of interest in developing formulations containing PTH (1-34) and its derivatives for the anabolic therapy of postmenopausal osteoporosis.

It has been shown that PTH (1-31), which stimulates adenylate cyclase but not PKC activity, is as potent as PTH(1-34) in its anabolic actions on the bone, but without stimulating resorption markers (Fraher, L. J. et al. 1999, J. Clin. Endocrino. Metab. 84:2739). There are many analogues of PTH (1-34) in which specific amino acids were replaced in order to increase the affinity to the receptor [[Nle8, 18, D-Trp12, Tyr34] bPTH (7-34): Rosen, H. N. et al. 1997, Calcif. Tissue. Int. 61:455-459] and reduce the oxidation upon storage [[Nle8, 18, Tyr34]-PTH (1-34)NH2: Noda, T. et al. 1980, The 41st Annual Meeting of Chemical Society of Japan, Abstr. No. 4S 12, Osaka].

Beta lactam derivatives of PTH(1-31) have also been produced and were shown to be effective in animal models of osteoporosis (Whitfield, J. F. et al. 1998, Calcif. Tissue Intl. 63:423; U.S. Pat. No. 6,316,410: Parathyroid hormone analogues for the treatment of osteoporosis). In addition to the injectable formulations of PTH (1-34), intranasal, oral and inhalatory formulations are currently being developed. The route of administration poses different challenges for formulating the same compound due to the differences in absorption, degradation, bioavailability, pharmacokinetics, the metabolite composition of the circulating peptides, immunogenicity and other parameters. Hence, either the molecule is modified, or formulations are prepared with the appropriate excipients so as to address the above-cited issues.

Transdermal delivery of peptides is an important administration route, due to the minimally-invasive nature of the procedures and the devices involved, as well as the ease of drug administration without the need for medical supervision. However, the issues facing transdermal delivery are very different as compared to other drug delivery systems, due to the special challenges posed by the skin barrier to peptides and more particularly of this barrier towards peptides in view of the presence of skin proteases.

Proteolytic degradation could lead to a loss of potency of PTH (in vitro and in vivo) and in particular in the case of shorter derivatives of PTH such as PTH (1-31).

There thus remains a need to develop PTH derivatives that are resistant to skin proteases, and which can be used for transdermal administration.

The present invention seeks to meet these and other needs.

The present description refers to a number of documents, the content of which is herein incorporated by reference in their entirety.

### SUMMARY OF THE INVENTION

Degradation by skin proteases poses certain problems to the bioavailability of peptides such as PTH, when administered transdermally. It was discovered In the present invention, that a degradation of PTH (1-34) takes place at the carboxyterminal end, due to the action of skin carboxypeptidases. In order to prevent such degradation, modified derivatives of PTH (1-34) were designed, synthesized and tested. These modified PTH (1-34) derivatives exhibit increased resistance to proteases in skin extracts, and manifest applicability in the transdermal delivery of these peptides to patients diagnosed with bone loss, or more particularly, patients susceptible to bone loss.

The present invention relates to protease-resistant analogues of biologically active derivatives of human PTH. In one particular embodiment of the present invention, the human PTH derivative is hPTH(1-34) comprising the following sequence:

H-Ser-Val-Ser-Glu-lle-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe-NHR (SEQ ID NO: 1)

wherein R is selected from the group consisting of a hydrogen atom, lower alkyl, lower alkenyl, substituted lower alkyl, substituted lower alkenyl, lower cycloalkylalkenyl, arylalkyl, substituted arylalkyl, lower arylalkyl, substituted lower arylalkyl, arylalkylenyl, substituted arylalkenyl and heteroarylalkenyl groups. R is more preferably selected from the group consisting of a hydrogen atom, a propyl group and a phenylpropyl group.

In another embodiment, the present invention relates to pharmaceutical compositions wherein a therapeutically effective amount of skin protease resistant PTH derivative (e.g. PTH (1-34)) is provided in an admixture with one or more physiologically-acceptable carriers or excipients.

The present invention also relates to pharmaceutical preparations wherein a therapeutically effective amount of a skin protease resistant PTH derivative as described in the present invention (e.g. PTH (1-34)), is present from about 10 μg to about 100 mg.

In addition, the present invention relates to the use of pharmaceutical preparations comprising a therapeutically effective amount of a skin protease resistant PTH derivative as described in the present invention (e.g. PTH (1-34)) in medical conditions such as osteoporosis, dental disease and malignancy in which bone loss is encountered or is susceptible of being encountered (e.g. in a patient predisposed to osteoporosis, for example). As well, the present invention relates to a method for treating or preventing diseases or conditions in which bone loss is encountered or is susceptible to being encountered, comprising the administration of a therapeutically effective amount of a skin protease resistant PTH derivative as described in the present invention (e.g. PTH (1-34)), together with one or more pharmaceutically suitable carriers or excipients.

In yet another embodiment of the present invention, the human PTH derivative is hPTH (1-31) comprising the following sequence:

H-Ser-Val-Ser-Glu-lle-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-NHR (SEQ ID NO: 2)

wherein R is selected from the group consisting of a hydrogen atom, lower alkyl, lower alkenyl, substituted lower alkyl, substituted lower alkenyl, lower cycloalkylalkenyl, arylalkyl, substituted arylalkyl, lower arylalkyl, substituted lower arylalkyl, arylalkylenyl, substituted arylalkenyl and heteroarylalkenyl groups. R is more preferably selected from the group consisting of a hydrogen atom, a propyl group and a phenylpropyl group.

In a further embodiment, the present invention relates to pharmaceutical preparations wherein a therapeutically effective amount of skin protease resistant PTH derivative (e.q. PTH (1-31)) is provided in an admixture with one or more physiologically-acceptable carriers or excipients.

In yet a further embodiment, the present invention relates to pharmaceutical preparations wherein a therapeutically effective amount of a skin protease derivative as described in the present invention (e.q. PTH (1-31)) is present from about 10 μg to about 100 mg.

Moreover, the present invention relates to the use of pharmaceutical preparations comprising a therapeutically effective amount of a skin protease resistant PTH derivative as described in the present invention (e.g. PTH (1-31)) in medical conditions such as osteoporosis, dental disease and malignancy in which bone loss is encountered or is susceptible of being encountered (e.g. in a patient predisposed to osteoporosis, for example). As well, the present invention relates to a method for treating or preventing diseases or conditions in which bone loss is encountered or is susceptible to being encountered, comprising the administration of a therapeutically effective amount of a skin protease resistant PTH derivative as described in the present invention (e.g. PTH (1-31)), together with one or more pharmaceutically suitable carriers or excipients.

Further scope and applicability will become apparent from the detailed description given hereinafter. It should be understood however, that this detailed description, while indicating preferred embodiments of the invention, is given by way of illustration only, since various changes and modifications within the spirit and scope of the invention will become apparent to those skilled in the art.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the proteolytic degradation of PTH (1-34), PTH (1-34) amide, PTH (1-34) propylamide, and PTH (1-34) NH-phenylpropyl in skin extracts. Aliquots (100 μg) of peptide were incubated at 37° C. with skin extract (1 mL) over the indicated periods of time. The remaining peptide was separated and quantified by HPLC. The data represent averages of duplicate experiments;

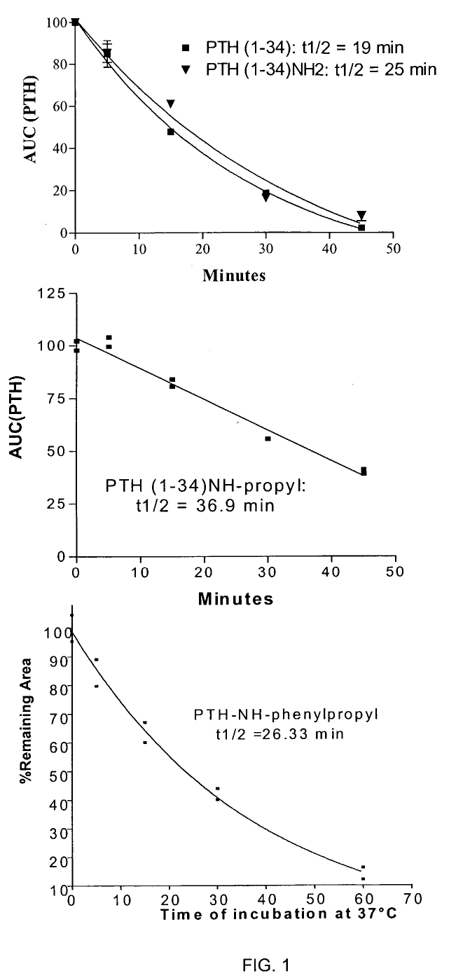

FIG. 2 shows HPLC chromatograms of PTH (1-34), PTH (1-34)NH2 and their major degradation products. The incubation of peptides (PTH(1-34), left panel and PTH(1-34) amide, right panel) with skin extracts, over the indicated times, was conducted as described in Example 2. Fractions containing the peaks were collected, lyophilized and the masses were determined by MALDI-TOF. PTH (1-34) eluted at t=25-26 minutes, whereas its major degradation product, having a mass corresponding to PTH(1-33), eluted at t=17 minutes. PTH(1-34) amide did not produce this metabolite following an incubation period of 30 min. Similar protection from skin proteases was observed for PTH (1-34) propylamide;

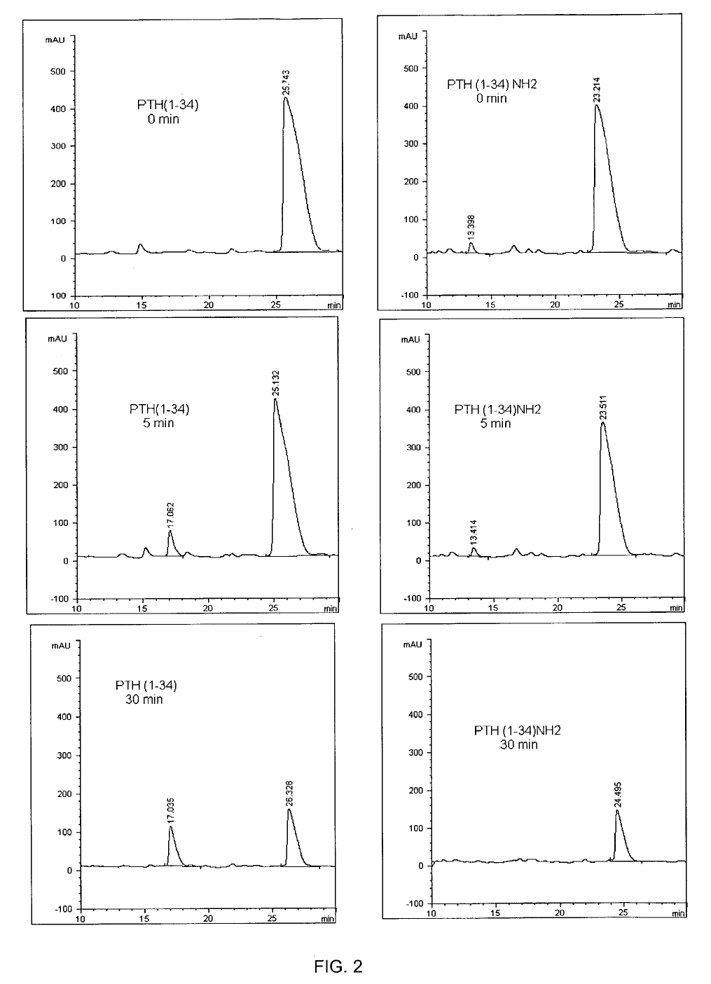

FIG. 3 is illustrative of cyclic AMP synthesis by PTH (1-34), PTH (1-34) amide and PTH (1-34) propylamide, in human osteoblastic cells. cAMP levels produced by Saos-2 cells in response to doses of PTH (1-34) and its analogues, were quantified by a radioimmuno assay kit.

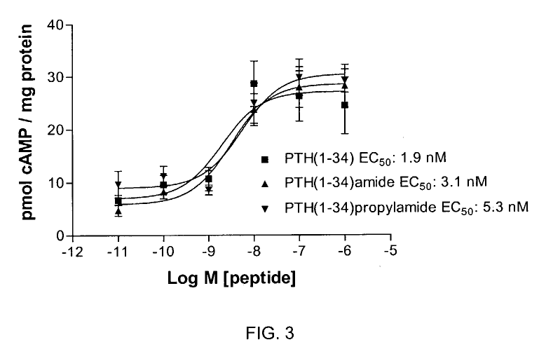

Other objects, advantages and features of the present invention will become more apparent upon reading of the following non-restrictive description of preferred embodiments with reference to the accompanying drawings, which is exemplary and should not be interpreted as limiting the scope of the present invention.

### DESCRIPTION OF THE PREFERRED EMBODIMENTS

In order to provide a clear and consistent understanding of the terms used in the present description, a number of definitions are provided below.

The term “acyl” is understood as being a group suitable for acylating a nitrogen atom to form an amide, carbamate, urea, amidine or guanidine, or an oxygen atom to form an ester group. Preferred acyl groups include benzoyl, acetyl, tert-butyl acetyl, para-phenyl benzoyl, and trifluoroacetyl. Preferred embodiments of acyl groups include acetyl and benzoyl. In a particularly preferred embodiment, the acyl group is acetyl.

The term “alkyl” is understood as being a saturated hydrocarbon chain having from about 1 to about 18 carbon atoms, preferably from about 1 to about 12, more preferably from about 1 to about 6, and more preferably still from about 1 to about 4 carbon atoms. Alkyl chains may be straight or branched. Preferred branched alkyl chains have one or two branches, more preferably one branch. Preferred alkyl chains are saturated. Unsaturated alkyl chains have one or more double bonds and/or one or more triple bonds. Preferred unsaturated alkyl chains have one or two double bonds or one triple bond, and more preferably one double bond. Alkyl chains may be unsubstituted or substituted, having from about 1 to about 4 substituents. Preferred alkyl chains are unsubstituted. Preferred substituted alkyl chains are mono-, di-, or trisubstituted. Preferred alkyl chain substituents include halo, haloalkyl, hydroxy, aryl (e.g., phenyl, tolyl, alkyloxphenyl, alkyloxycarbonylphenyl, halophenyl), heterocyclyl, and heteroaryl.

The term “alkenyl” refers to a straight or branched hydrocarbon chain from 2 to 12 carbon atoms including at least one double bond. Examples include, but are not limited to ethenyl, allyl, and 2- or 3-butenyl.

The term “alkynyl” refers to a straight or branched hydrocarbon chain from 2 to 12 carbon atoms including at least one triple bond. Examples include, but are not limited to ethynyl, 2-propynyl, and 2- or 3-butynyl.

The term “lower alkyl” refers to straight or branched chain radicals having up to four carbon atoms. Examples include, but are not limited to methyl, ethyl, n-propyl, isopropyl, n-butyl, isobutyl, sec-butyl and tert-butyl.

The term “lower alkenyl” refers to straight or branched chain radicals having three or four carbon atoms and having one double bond. Examples include, but are not limited to allyl, cis- or trans-but-2-enyl, cis- or trans-but-3-enyl, and 2-methylallyl.

The term “alkylene” refers to divalent straight or branched chain radicals having up to seven carbon atoms. Examples include, but are not limited to —(CH₂)₂—, —(CH₂)₃—, —(CH₂)₄—, —CH₂CH(CH₃)—.

The terms “cycloalkyl” and cycloalkenyl” refer to saturated and unsaturated cyclic and bicylic radicals having 3 to 12 carbon atoms and which may be optionally substituted. Representative examples of such groups are cyclopropyl, cyclobutyl, cyclopentyl, cyclohexyl, norbornyl, and the like.

The terms “lower cycloalkyl” and “lower cycloalkenyl” refer to saturated and unsaturated cyclic and bicylic radicals having 3 to 6 carbon atoms and which may be optionally substituted.

The term “cycloalkylalkenyl” refers to a cycloalkyl as defined above and alkenyl as defined above.

The term “lower cycloalkylalkenyl” refers to lower cycloalkyl as defined above and alkenyl as defined above. Representative examples of such groups are cis or trans 3-cyclohexylprop-2-enyl, cis or trans 4-cyclohexylbut-3-enyl, cis or trans 4-cyclohexylbut-2-enyl, cis or trans 3-cyclopentylprop-2-enyl, cis or trans 4-cyclopentylbut-3-enyl, cis or trans 4-cyclopentylbut-2-enyl, cis or trans 3-cyclopropylprop-2-enyl, cis or trans 4-cyclopropylbut-3-enyl, cis or trans 4-cyclopropylbut-2-enyl and the like.

The term “aryl” refers to phenyl, 1-naphthyl, 2-naphthyl.

The term “arylalkyl” refers to an aryl group appended to an alkyl radical. Representative examples of such a group are phenylmethyl, 1-phenylethyl, 1-phenylpropyl, 1-naphthylethyl and the like.

The term “lower arylalkyl” refers to an aryl group appended to a lower alkyl radical.

The term “substituted arylalkyl” refers to an arylalkyl group as defined above wherein the aryl group is substituted as defined for “substituted aryl”.

The term “substituted lower arylalkyl” refers to a lower arylalkyl group as defined above wherein the aryl group is substituted as defined for “substituted aryl”.

The term “arylalkenyl” refers to an aryl group appended to an alkenyl radical. Representative examples of such a group are phenylethenyl, 1-naphthylethenyl and the like.

The term “substituted arylalkenyl” refers to an arylalkenyl group as defined wherein the aryl group is substituted as defined for “substituted aryl”. Representative examples of such groups are cis or trans 3[4-methoxyphenyl]-prop-2-enyl, cis or trans 3[4-fluorophenyl]-prop-2-enyl, cis or trans 4[4-methoxyphenyl]-but-2-enyl, cis or trans 4[4-methoxyphenyl]-but-3-enyl, cis or trans 4[2,6-dichlorophenyl]-but-3-enyl and the like.

The term “arylalkylenyl” refers to an aryl group appended to an alkylenyl radical as defined above. Representative examples of such groups are 1-indanyl; 2-indanyl; 1,2,3,4-tetrahydro-naphthalen-1-yl; and 1,2,3,4-tetrahydro-naphthalen-2-yl and the like.

The term “heterocyclyl” refers to a group comprised of atoms selected from carbon, nitrogen, oxygen and sulfur atoms, necessary to complete a 5- or 6-membered heterocyclic ring. Examples include, but are not limited to pyrrolyl, oxazolyl and pyridyl.

The term “heteroaryl” refers to unsaturated rings of five or six atoms containing one or two O- and/or S-atoms and/or one to four N-atoms, provided that the total number of heteroatoms in the ring is four or less. The heteroaryl ring is attached by way of an available carbon or nitrogen atom. Preferred heteroaryl groups are 2-, 3-, or 4-pyridyl, 4-imidazolyl, 4-thiazolyl, 2- and 3-thienyl, 2- and 3-furyl. The term heteroaryl also includes bicyclic rings wherein the 5- or 6-membered ring containing O, S and N-atoms, as defined above, is fused to a benzene or pyridyl ring. Preferred bicylic rings are 2- and 3-indolyl as well as 4- and 5-quinolinyl. The mono- or bicyclic heteroaryl ring can also be additionally substituted at an available carbon atom by a substituent selected from the group consisting of lower alkyl, halo, hydroxy, benzyl and cyclohexylmethyl. Additionally, if the mono- or bicylic ring has an available N-atom, then such a nitrogen atom can also be substituted by one of the N-protecting groups as for example, but not limited to, an N-benzyloxycarbonyl, N-tosyl, N-lower alkyl, and N-benzyl. Non-limiting examples of other such protecting groups can be found in reference manuals such as for example Green and Wuts (Protective Groups in Organic Synthesis, John Wiley & Sons, New York, N.Y., 1991).

The term “heteroarylalkenyl” refers to a heteroaryl group appended to an alkenyl radical. Representative examples of such groups are cis or trans-4-(1H-indol-3-yl)-but-2-enyl, cis or trans-3-pyridin-2-yl-prop-2-enyl, cis or trans-4-pyridin-2-yl-but-3-enyl, cis or trans-3-(1H-imidazol-2-yl)-prop-2-enyl, cis or trans-3-thiophen-3-yl-prop-2-enyl, cis or trans-3-furan-3-yl-prop-2-enyl and the like.

The term “substituted lower alkyl” refers to such straight or branched chain radicals of up to four carbon atoms, wherein one or more, preferably one or two hydrogens atoms, have been replaced by a hydroxy, amino, cyano, halo, trifluoromethyl, —NH(lower alkyl), —N(lower alkyl)₂, lower alkoxy, lower alkylthio or carboxy substituent.

The term “lower alkoxy and lower alkylthio” refers to such lower alkyl groups as defined above attached to an oxygen or sulfur atom.

The term “substituted lower alkenyl” refers to such straight or branched chain radicals composed of three to four carbon atoms having a double bond, and wherein a hydrogen atom is replaced by a hydroxy, amino, halo, trifluoromethyl, cyano, —NH(lower alkyl), —N(lower alkyl)₂, lower alkoxy, lower alkylthio or carboxy group.

The terminology “substituted aryl” refers to phenyl, 1-naphthyl and 2-naphthyl having a substituent selected from the group consisting of lower alkyl, lower alkoxy, lower alkylthio, halo, hydroxy, trifluoromethyl, amino, —NH(lower alkyl), and —N(lower alkyl)₂ substituents; as well as di- and tri-substituted phenyl, 1-naphthyl and 2-naphthyl, wherein the substituents are selected from the group consisting of methyl methoxy, methylthio, halo, hydroxy and amino substituents.

The term “phenyl” refers to a six-membered monocyclic aromatic ring which may or may not be substituted. It may have from about 1 to about 4 substituents that may be located at the ortho, meta or para position of the phenyl ring, or any combination thereof. Preferred phenyl substituents include halogen, cyano, alkyl, heteroalkyl, haloalkyl, phenyl, phenoxy or any combination thereof. More preferred phenyl substituents include halogen and haloalkyl. In a preferred embodiment the phenyl substituent is a halogen. The preferred substitution pattern of the phenyl ring consists of the ortho or meta positions. The most preferred substitution pattern of the phenyl ring consists of the ortho position.

As used herein “pharmaceutically acceptable carrier” or “excipient” includes any and all solvents, dispersion media, coatings, antibacterial and antifungal agents, isotonic and absorption delaying agents, and the like, that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions, and sterile powders for the extemporaneous preparation of sterile injectable solutions or dispersions. The use of such media and agents with pharmaceutically active substances is well known in the art. Unless a conventional medium or agent is incompatible with the active compound, use thereof in the pharmaceutical compositions of the present invention is contemplated. Supplementary active compounds can also be incorporated into the compositions. The terminology “pharmaceutically acceptable carrier or excipient” is well-known in the art. It can be adapted by a person of ordinary skill to meet particular needs. Non-limiting examples of such carriers or excipients can be found for example in Remington (Pharmaceutical Science, 16th Ed., Mack Ed.). In a particularly preferred embodiment, the carrier or excipient is chosen for transdermal application of a PTH derivative of the present invention.

Effective Dose (or Effective Amount)

The toxicity and therapeutic efficacy of PTH derivatives, such as for example the LD₅₀ (Lethal Dose to 50% of the population) and the ED₅₀ (therapeutically effective dose in 50% of the population) can be determined by standard pharmaceutical procedures in experimental animals. The dose ratio between toxic and therapeutic effects is the therapeutic index, and which can be expressed as the LD₅₀/ED₅₀ ratio. Compounds that exhibit large therapeutic indices are preferred. The dosage of such compounds preferably lies within a range of circulating concentrations that include the ED₅₀, but with little or no toxicity. The dosage may vary within this range, depending on the dosage form employed and the route of administration utilized. A dose may be formulated in animal models in order to obtain a circulating plasma concentration range that includes the IC₅₀ (the concentration of the test compound which achieves a 50% inhibition of the symptoms) as determined in in vitro and ex vivo assays as well as in animal studies. Such information can then be used to more accurately determine useful doses in humans.

Plasma levels of the PTH derivatives may be measured, for example, by high performance liquid chromatography (HPLC). The effective dose of a PTH derivative of the present invention could be 0.01 micrograms to 100 mg/Kg and is determined by the route of administration, pharmaceutical preparation and the mode of delivery.

Formulation and Use

Pharmaceutical compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation (either through the mouth or the nose), oral, buccal, parenteral or rectal administration. Techniques and formulations may generally be found in “Reminington's Pharmaceutical Sciences”, (Meade Publishing Co., Easton, Pa.). For topical administration, the PTH derivatives of the present invention are formulated into solutions, ointments, salves, gels, or creams as generally known in the art. Therapeutic compositions must typically be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), or suitable mixtures thereof. Proper fluidity can be maintained, for example, by the use of a coating such as lecithin, by the maintenance of the required particle size (in the case of a dispersion), and by the use of surfactants. In many cases it will be preferable to include isotonic agents, such as for example, sugars, polyalcohols such as mannitol or sorbitol, or sodium chloride, in the composition. Prolonged absorption of the injectable compositions can be brought about by including an agent in the composition that delays absorption, such as for example monostearate salts and gelatin. Moreover, the PTH derivatives of the present invention can be administered as a time release formulation, such as for example in a composition including a slow release polymer. The PTH derivatives can be formulated with carriers protecting the compound against rapid release, such as is observed with controlled release formulations, including implants and micro-encapsulated delivery systems. Biodegradable and biocompatible polymers can be used, such as for example ethylene vinyl acetate, polyanhydrides, polyglycolic acid, collagen, polyorthoesters, polylactic acid and polylactic-polyglycolic copolymers (PLG). Many methods for the preparation of such formulations are patented or generally known to those skilled in the art.

Sterile injectable solutions can be prepared by incorporating the active compound (PTH derivative) in the required amount in an appropriate solvent with one or a combination of ingredients as enumerated above and as required, followed by filtered sterilization. Dispersions are generally prepared by incorporating the active compound in a sterile vehicle containing a basic dispersion medium and the required other ingredients (selected from those enumerated above). In the case of sterile powders, and for the preparation of sterile injectable solutions, the preferred methods of preparation are vacuum drying and freeze-drying. This yields a powder of the active ingredient and of any additional desired ingredient obtained from a previously sterile-filtered solution.

The PTH derivatives of the present invention may be formulated with one or more additional compounds enhancing their solubility. Of course, it might be suitable to mix more than one protease resistant peptide of the present invention, with one or more pharmaceutically acceptable carriers or excipients. Additionally, the therapeutic compositions of the present invention, comprising a PTH derivative, may be provided in containers or commercial packages that contain user instructions, for the prevention and/or treatment of bone loss experienced by patients diagnosed with medical conditions such as osteoporosis, dental disease and malignancy.

Although the present invention is particularly exemplified with hPTH (1-34) derivatives, the present invention is not so limited. Indeed, a hPTH derivative which comprises a sufficient portion of the hPTH sequence to retain biological activity in vitro and especially in vivo is encompassed by the present invention. In essence therefore, the present invention relates to skin protease resistant hPTH derivatives which comprise at least amino acid (aa) 1-14, more particularly at least aa 1-28, and even more particularly at least aa 1-31 of the serum-clipped hPTH sequence (1-38). Non-limiting examples of such derivatives include (1-14), (1-15), (1-28), (1-29), (1-32), (1-33), (1-35), (1-36), (1-37), and (1-38) hPTH derivatives.

While the present invention has been exemplified using amide protecting groups at the C-terminal end of a chosen hPTH sequence, the present invention is not so limited. Indeed, a person of ordinary skill can use other types of protecting groups to protect the C-terminal portion of a hPTH derivative. Such person of ordinary skill would chose, amongst the possible protecting groups, the ones that at least transiently protect the PTH derivative from C-terminal skin protease, while not significantly affecting the biological activity of the protected hPTH derivative. Numerous protecting groups are known in the art. Non-limiting examples of such protecting groups that could be used in the present invention include ester groups and phosphate groups. An example of a reference in which examples of protecting groups can be found include “Protective Groups in Organic Synthesis”, (Greene, T. W.; Wuts, P. G. M., John Wiley & Sons, Inc.: 1991, New York).

In certain situations, it will be understood that it might be advantageous to additionally protect the amino terminal end of the C-terminally protected hPTH derivatives of the present invention.

The present invention is illustrated in further detail by the following non-limiting examples.

### EXAMPLE 1

### Synthesis and Characterization of PTH (1-34) Derivatives

hPTH(1-34) and its derivatives were prepared by solid phase synthesis using Fmoc chemistry (Fmoc Solid Phase Peptide Synthesis. A Practical Approach; Chan, W. C. and White, P. D., 2000, Oxford University Press, New York, USA, p346), on an in-house manual peptide synthesizer. Fmoc-Phe-Wang resin (0.9 mmol/g) (home-made) was used as the starting material for hPTH(1-34). Aminomethyl resin (0.9 mmol/g) was used for the hPTH(1-34)amide, and Fmoc-Phe-2-chlorotrityl resin was used for hPTH(1-34) propylamide. For coupling of all the amino acids, 3 equivalents of amino acids, 3 equivalents of BOP and 6 equivalents of DIEA were used. The coupling time was 60 minutes. hPTH(1-34) and the hPTH(1-34)amide were cleaved using a TFA cocktail (92% TFA, 2% ethanedithiol, 2% thioanisole, 2% triisopropylsilane, 2% water), followed by 2% (w/v) phenol, for 2 hours. For the hPTH(1-34) propylamide, the peptide (Fmoc-hPTH(1-34)) was cleaved from the resin with AcOH/TFE/DCM (2:2:6) for 2 hours. It was then converted into propylamide using 3 equivalents of propylamine, 3 equivalents of BOP and 6 equivalents of DIEA. The Fmoc at the N-terminus was removed and the peptide was fully deprotected (lateral chains) using a TFA cocktail (92% TFA, 2% ethanedithiol, 2% thioanisole, 2% triisopropylsilane, 2% water), followed by 2% (w/v) phenol), for 2 hours. The crude peptides were precipitated, washed and triturated with ethyl ether and stored at −20° C. until their purification.

All the analogs were purified by reverse-phase HPLC, using a Prep LC 4000 system from Waters, using a TFA/Acetonitrile gradient, a Vydac column (30×2.5 cm, C18, 15/20 μm, 300Å) or a EKA column (50.8 mm×250 mm, C18, 10 μm, 100Å), 229 nm, 40 ml/min. The analogs were analyzed by analytical HPLC using an Agilent 1100 Series with a TFA/Acetonitrile gradient, a Zorbax column (250×4.6 mm, SBC8, 5 μm, 100Å) or a Waters column (150×3.9 mm, C18, 5 μm, 100Å), 214 nm, 1 ml/min. The analogs were converted into an acetate salt using an ion exchange Amberlite resin. The final derivatives were analyzed by MALDI-TOF MS (Voyager-DE Perseptive Biosystems) according to the manufacture's procedure.

### EXAMPLE 2

### Degradation of PTH (1-34) Derivatives in Hairless Guinea Pig Skin Extracts

Sample Preparation Method:

Hairless guinea pig skin extracts (1 mL aliquots) were prepared from samples of plasma and skin extracts, respectively, and stored at about −20° C. Stock solutions of hPTH(1-34), hPTH(1-34)NH2, hPTH (1-34)NH-propyl, and hPTH (1-34)NH-phenylpropyl were prepared in HPLC-grade water to achieve a concentration of 10 mg of peptide/mL. Aliquots of skin extracts (1 ml) were incubated at 37° C. for 10 minutes prior to the addition of peptide (100 μg; 10 μL stock solution) or water (10 μL) (control). The samples were maintained at 37°C. for 0, 5, 15, 30 and 45 minutes. Upon the completion of the incubation, the samples were quenched with trifluoroacetic acid (240 μL; 1M). The tubes were mixed and placed on ice for 10 minutes, after which aqueous TFA (250 μL; 0.05%) was added to each tube. The tubes were mixed again and centrifuged at 3000 g for 20 minutes at 40° C. The supernatants were transferred to new tubes and placed on ice pending solid phase extraction using Sep-Pak C18 cartridges (1 cc, 100 mg, Waters). The cartridges were washed with 1 ml of an aqueous solution of 0.05% TFA in 80% (v/v) acetonitrile, followed by 1 ml of 0.05% TFA in water. Samples were loaded onto the SPE cartridges and the cartridges washed five times with 1 ml of 0.05% TFA in water. Retained material was eluted with two additions of 1 ml 0.05% TFA in 80% (v/v) acetonitrile. The eluates were frozen in liquid nitrogen and lyophilized overnight.

HPLC Analysis:

The lyophilized samples were resuspended in 200 μL of 0.1 N acetic acid and centrifuged at 13,000 g for 15 minutes at room temperature. The following equipment and conditions were used; Agilent 1100 HPLC system, UV detector wavelength: 214 nm; Guard column cartridges: C18 ODS; 4.0×3.0 mm (Phenomenex)Column: Sephasil C18 reverse-phase Peptide; 250×4.6 mm, 5 mm, 100 Å (Amersham Pharmacia Biotech), autosampler & column temperature: 22-24° C., Mobile phase A: 0.05% TFA, Mobile phase B: 0.04% TFA in acetonitrile, Injection volume: 100 μl, Flow Rate: 1 ml/min, gradient: 27%-34% of B over 50 min. Fractions containing the metabolites of PTH derivatives were collected, concentrated by lyophilization and their masses determined by MALDI-TOF MS (using Voyager-DE Perseptive Biosystems according to the manufacture's procedure).

### EXAMPLE 3

### Cyclic AMP Synthesis Assay

Cell culture: Saos-2 cells (ATCC# HTB-85) were propagated in McCoy's 5a medium supplemented with 15% fetal bovine serum, 10U/ml penicillin, 10 ug/ml streptomycin and 0.5 ug/ml fungizone, under a humidified atmosphere of 5% CO₂ at 37° C. Prior to the experiment, cells were subcultured in 12-well plates at a density of 100 000 cells/well (dose-response) or in 24-well plates at a density of 50 000 cells/well (competition binding). After 48 hours of incubation, the media was replaced for a complete media containing 1 uM dexamethasone and the incubation pursued for a further 24 hours.

The cells were washed twice with PBS and then incubated with 500 μl/well of HBBS (20 mM Hepes pH 7.2, 118 mM NaCl, 4.6 mM KCl, 1 mM CaCl₂, 10 mM D-Glucose and 100 uM 3-isobutyl-1-methylxanthine) at 37° C. After 15 minutes of incubation, the media was replaced for 150 μl HBBS containing the different PTH analogues. The incubation was pursued for 40 minutes at 37° C. and the plate rocked once every 10 minutes. At the end of the incubation, 100 μl of cell supernatant was measured by radioimmunoassay using a commercial kit (Diagnostic Products Corporation Inc.). The data were analyzed using GraphPad Prism version 3.02 for Windows, GraphPad Software, San Diego Calif. USA.

Although the present invention has been described hereinabove by way of preferred embodiments thereof, it can be modified without departing from the spirit and nature of the subject invention as defined in the appended claims.

1. A peptide comprising the following sequence:
H-Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe-NHR
wherein R is selected from the group consisting of a hydrogen atom, lower alkyl, lower alkenyl, substituted lower alkyl, substituted lower alkenyl, lower cycloalkylalkenyl, arylalkyl, substituted arylalkyl, lower arylalkyl, substituted lower arylalkyl, arylalkylenyl, substituted arylalkenyl and heteroarylalkenyl groups:

2. A peptide as defined in claim 1, wherein R is a hydrogen atom.

3. A peptide as defined in claim 1, wherein R is propyl.

4. A peptide as defined in claim 1 wherein R is phenylpropyl.

5. An admixture comprising the peptide as defined in any one of claims 1 to 4 and a or more pharmaceutically acceptable carrier or excipients.

6. A pharmaceutical composition comprising from about 10 μg to about 100 mg of the peptide as defined in any one of claims 1 to 4 and a or more pharmaceutically acceptable carriers or excipients.

7. A method for treating or preventing diseases or conditions involving loss of bone mineral, comprising administering a therapeutically effective amount of the pharmaceutical composition as defined in claim 6 to a patient in need thereof.

8. The method of claim 7, wherein said diseases or conditions are selected from the group consisting of dental disease, osteoporosis and malignancy.

9. The method as defined in claim 8, wherein the loss of bone mineral is due to dental disease.

10. The method as defined in claim 8, wherein the loss of bone mineral is due to osteoporosis.

11. The method as defined in claim 8, wherein the loss of bone mineral is due to malignancy.

12. The method as defined in any one of claims 7 to 11, wherein said pharmaceutical composition effects replacement of bone mineral.

13. A peptide comprising the following sequence:
H-Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-NHR
wherein R is selected from the group consisting of a hydrogen atom, lower alkyl, lower alkenyl, substituted lower alkyl, substituted lower alkenyl, lower cycloalkylalkenyl, arylalkyl, substituted arylalkyl, lower arylalkyl, substituted lower arylalkyl, arylalkylenyl, substituted arylalkenyl and heteroarylalkenyl groups.

14. A peptide as defined in claim 13, wherein R is a hydrogen atom.

15. A peptide as defined in claim 13, wherein R is propyl.

16. A peptide as defined in claim 13, wherein R is phenylpropyl.

17. An admixture comprising the peptide as defined in any one of claims 13 to 17 and a or more pharmaceutically acceptable carriers or excipients.

18. A pharmaceutical composition comprising from about 10 μg to about 100 mg of the peptide as defined in any one of claims 13 to 17 and a or more pharmaceutically acceptable carriers or excipients.

19. A method for treating or preventing diseases or conditions involving loss of bone mineral, comprising administering a therapeutically effective amount of the pharmaceutical composition as defined in claim 18 to a patient in need thereof.

20. The method of claim 19, wherein said diseases or conditions are selected from the group consisting of dental disease, osteoporosis and malignancy.

21. The method as defined in claim 19, wherein the loss of bone mineral is due to dental disease.

22. The method as defined in claim 19, wherein the loss of bone mineral is due to osteoporosis.

23. The method as defined in claim 19, wherein the loss of bone mineral is due to malignancy.

24. The method as defined in any one of claims 19 to 23, wherein said pharmaceutical composition effects replacement of bone mineral.

25. A peptide as defined in claim 1, consisting of the following sequence:
H-Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe-NHR

26. A peptide as defined in claim 13, consisting of the following sequence:
H-Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-NHR


SEQUENCE LISTING (2 sequences)

SEQ ID NO: 1 - protein, 34 aa, Artificial Sequence
  Description of Artificial Sequence  Synthetic Peptide
SVSEIQLMHNLGKHLNSMERVEWLRKKLQDVHNF

SEQ ID NO: 2 - protein, 31 aa, Artificial Sequence
  Description of Artificial Sequence  Synthetic Peptide
SVSEIQLMHNLGKHLNSMERVEWLRKKLQDV